(19) United States
(12) Patent Application Publication
(10) Pub. No.: US 2017/0096634 A1
(43) Pub. Date: Apr. 6, 2017

(54) MYCOBACTERIA PRE-ANALYTIC REAGENT

(71) Applicant: Roche Molecular Systems, Inc., Pleasanton, CA (US)

(72) Inventors: Rochak Mehta, Fremont, CA (US); Jenny A. Johnson, Castro Valley, CA (US)

(21) Appl. No.: 15/281,724

(22) Filed: Sep. 30, 2016

Related U.S. Application Data
(60) Provisional application No. 62/236,612, filed on Oct. 2, 2015.

(51) Int. Cl. C12N 1/20

(52) U.S. Cl. CPC C12N 1/20

(57) ABSTRACT

A pre-analytic reagent is provided for liquefaction of a sputum sample and complete inactivation of one or more Mycobacteria, e.g., Mycobacterium tuberculosis (MTB) suspected of being contained in the sputum sample at room temperature. The pre-analytic reagent may include one or more of each of: a chaotropic agent, a chelator, a reducing agent, a detergent, and acetic acid. The pre-analytic reagent can be configured to be effective to liquefy the sputum sample and completely inactivate the Mycobacteria suspected to be contained therein.

### CROSS-REFERENCE TO RELATED INVENTION

This application claims the benefit of priority of U.S. Provisional Patent Application Ser. No. 62/236,612, filed Oct. 2, 2015, which is incorporated herein by reference in its entirety.

### FIELD OF THE INVENTION

The present disclosure relates to the field of pre-analytic reagents, particularly to a Mycobacteria pre-analytic reagent, methods, and kits for a sputum sample and inactivation (i.e., killing) of one or more Mycobacteria suspected of being contained in the sputum sample.

### BACKGROUND OF THE INVENTION

The isolation of biological materials such as nucleic acids or proteins from complex biological mixtures such as clinical samples is of considerable significance especially for diagnostic purposes. When working with certain types of samples, such as sputum, the isolation of such biological material can be problematic. Sputum is generally made of inflammatory exudate from the lower respiratory tract mixed with saliva. For example isolation of Mycobacteria, such as Mycobacterium tuberculosis (MTB), Mycobacterium avium, and/or Mycobacterium intracellulare from a sample of sputum can be particularly problematic because sputum is a viscous sample type and liquefaction of the sputum is required before sample preparation for testing.

MTB poses potential risks for the laboratory personnel working with this microorganism (Kao et al., J Clin Microbiol, 1997, 1847-1851). There are several reports of laboratory-acquired tuberculosis infections, with aerosols and skin punctures being the most common reported routes of transmission (Menzies et al., New England J Med, 1995, 322(2)-92-98). While diagnostic samples of MTB can be manipulated under biosafety level 2 (BSL2) conditions, live cultured MTB organisms should be manipulated under BSL3 conditions to ensure laboratory safety. Accordingly, MTB organisms have to be inactivated prior to release outside a BSL3 laboratory for further molecular biology manipulation. This emphasizes the need for complete inactivation of MTB before downstream sample processing and PCR amplification.

Several methods have been developed for liquefaction of sputum and inactivation of Mycobacteria, however such methods do not accomplish both liquefying sputum and complete inactivation of the Mycobacteria present at a high concentration in a single-step method performed at room temperature, which is addressed by the present disclosure.

### SUMMARY OF THE INVENTION

In one embodiment, a method is provided for liquefaction of a sputum sample and inactivation of Mycobacteria suspected of being contained therein, the method including the steps of contacting a sputum sample suspected of containing Mycobacteria with an amount of a composition effective to liquefy the sputum sample and completely inactivate the Mycobacteria at room temperature. The composition may include one or more of each of: a chaotropic agent, a chelator, a reducing agent, a detergent, and acetic acid. In some embodiments, a method for liquefaction of a sputum sample and inactivation of Mycobacteria suspected of being contained therein is provided comprising contacting a sputum sample suspected of containing Mycobacteria with an amount of a composition effective to liquefy the sputum sample and to completely inactivate the Mycobacteria at room temperature, wherein the composition comprises a chaotropic agent, a chelator, a reducing agent, a detergent, and acetic acid. In certain embodiments, the chaotropic agent is present in an amount from about 0.5 M to about 6 M; the chelator is present in an amount from about 0.01 mM to about 400 mM; the reducing agent is present in an amount from about 130 mM to about 650 mM; the detergent is present in an amount from about 0.2% to about 15%; and the acetic acid is present in an amount from about 6% to about 11%. In some embodiments, the chaotrope is guanidine thiocyanate (GuSCN). In some embodiments, the chelator is sodium citrate. In some embodiments, the reducing agent is Dithiothreitol (DTT). In some embodiments, the detergent is polydocanol and Na N-lauroyl sarcosine. In some embodiments, the Mycobacteria is Mycobacterium tuberculosis (MTB). In some embodiments, the sample is contacted with the composition at room temperature for about 15 minutes to about 2 hours. In some embodiments, the sample is mixed with the pre-analytic reagent in a 1:1 ratio (v/v).

In another embodiment, a pre-analytic reagent is provided for liquefaction of a sputum sample and inactivation of one or more Mycobacteria, e.g., Mycobacterium tuberculosis (MTB) suspected of being contained in the sputum sample. The pre-analytic reagent may include one or more of each of: a chaotropic agent, a chelator, a reducing agent, a detergent, and acetic acid. The pre-analytic reagent can be configured to be effective to liquefy the sputum sample and completely inactivate the Mycobacteria suspected to be contained therein at room temperature. In some embodiments, a pre-analytic reagent for liquefaction of a sputum sample and inactivation of one or more Mycobacteria suspected of being contained therein is provided that comprises a chaotropic agent, a chelator, a reducing agent, a detergent, and acetic acid, wherein the pre-analytic reagent is effective to liquefy the sputum sample and completely inactivate the Mycobacteria at room temperature. In certain embodiments, the chaotropic agent is present in an amount from about 0.5 M to about 6 M; the chelator is present in an amount from about 0.01 mM to about 400 mM; the reducing agent is present in an amount from about 130 mM to about 650 mM; the detergent is present in an amount from about 0.2% to about 15%; and the acetic acid is present in an amount from about 6% to about 11%. In some embodiments, the chaotrope is guanidine thiocyanate (GuSCN). In some embodiments, the chelator is sodium citrate. In some embodiments, the reducing agent is Dithiothreitol (DTT). In some embodiments, the detergent is polydocanol and Na N-lauroyl sarcosine. In some embodiments, the Mycobacteria is Mycobacterium tuberculosis (MTB).

In another embodiment, a kit is provided including a composition effective to liquefy a sputum sample and completely inactivate Mycobacteria suspected to be contained therein at room temperature. The composition may include one or more of each of: a chaotropic agent, a chelator, a reducing agent, a detergent, and acetic acid. In some embodiments, the kit comprises a composition effective to liquefy a sputum sample and completely inactivate Mycobacteria suspected to be contained therein at room temperature, wherein the composition comprises a chaotropic agent, a chelator, a reducing agent, a detergent, and acetic acid. In certain embodiments, the chaotropic agent is present in an amount from about 0.5 M to about 6 M; the chelator is present in an amount from about 0.01 mM to about 400 mM; the reducing agent is present in an amount from about 130 mM to about 650 mM; the detergent is present in an amount from about 0.2% to about 15%; and the acetic acid is present in an amount from about 6% to about 11%. In some embodiments, the chaotrope is guanidine thiocyanate (GuSCN). In some embodiments, the chelator is sodium citrate. In some embodiments, the reducing agent is Dithiothreitol (DTT). In some embodiments, the detergent is polydocanol and Na N-lauroyl sarcosine. In some embodiments, the Mycobacteria is Mycobacterium tuberculosis (MTB). In some embodiments, the kit further comprises instructions for effectively liquefying a sputum sample and completely inactivating Mycobacteria. In some embodiments, the instructions indicate that the sample is to be contacted with the composition at room temperature for about 15 minutes to about 2 hours. n some embodiments, the instructions indicate that the sample is to be mixed with the pre-analytic reagent in a 1:1 ratio (v/v). In some embodiments, the composition is provided in a container. In some embodiments, the kit further comprises a pipette. In some embodiments, the pipette may be a disposable pipette. In some embodiments, the kit further comprises at least one component for performing a polymerase chain reaction, said components being selected from nucleoside triphosphates, nucleic acid polymerase, and buffers necessary for the function of the nucleic acid polymerase.

Unless otherwise defined, all technical and scientific terms used herein have the same meaning as commonly understood by one of ordinary skill in the art to which this subject matter belongs. Although methods and materials similar or equivalent to those described herein can be used in the practice or testing of the present disclosure, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the present subject matter are set forth in the accompanying drawings and the description below. Other features, objects, and advantages of the disclosure will be apparent from the drawings and detailed description, and from the claims.

### DETAILED DESCRIPTION OF THE INVENTION

As mentioned above, one embodiment of the present disclosure is directed to a pre-analytic reagent is provided for liquefaction of a sputum sample and inactivation (i.e., killing) of one or more Mycobacteria, e.g., Mycobacterium tuberculosis (MTB) suspected of being contained in the sputum sample at room temperature. The pre-analytic reagent may include one or more of each of: a chaotropic agent, a chelator, a reducing agent, a detergent, and acetic acid. The pre-analytic reagent can be configured with proper concentrations of each ingredient to be effective to liquefy the sputum sample and completely inactivate the Mycobacteria suspected to be contained therein at room temperature.

The term “liquefaction” as used herein in the context of sputum samples means the act or operation of making or becoming liquid.

The presently disclosed pre-analytic reagent composition may include one or more chaotropic agents which may be present in an amount from about 0.5 M to about 6 M, for example from about 1 M to about 5 M, for example from about 2.5 M to about 4.5 M, for example about 4 M. The chaotropic agent may be any chaotropic agent suitable for the purpose intended herein. In some embodiments, the chaotropic agent may include guanidine thiocyanate (GuSCN), guanidine hydrochloride (GuHCl), guanidine isothionate, potassium thiocyanate (KSCN), sodium iodide, sodium perchlorate, urea, or any combination thereof.

The pre-analytic reagent composition may include one or more chelators which may be present in an amount from about 0.01 mM to about 400 mM, for example from about 10 mM to about 300 mM, for example from about 50 mM to about 400 mM, for example about 50 mM. The chelator may be any chelator suitable for the purpose intended herein. In some embodiments, chelators may include ethylene glycol tetraacetic acid (EGTA), hydroxyethylethylenediamine-triacetic acid (HEDTA), diethylene triamine pentaacetic acid (DTPA), N,N-bis(carboxymethyl) glycine (NTA), ethylenediaminetetraacetic (EDTA), citrate anhydrous, sodium citrate, calcium citrate, ammonium citrate, ammonium bicitrate, citric acid, diammonium citrate, potassium citrate, magnesium citrate, ferric ammonium citrate, lithium citrate, sodium acetate, or any combination thereof.

The pre-analytic reagent composition may include one or more reducing agents which may be present in an amount from about 100 mM to about 650 mM, for example from about 110 mM to about 600 mM, for example from about 120 mM to about 650 mM, for example about 130 mM. The reducing agent may be any reducing agent suitable for the purpose intended herein. In some embodiments, the reducing agent may include 2-mercaptoethanol (βME), tris(2-carboxyethyl)phosphine (TCEP), dithiothreitol (DTT), formamide, dimethylsulfoxide (DMSO), or any combination thereof.

The pre-analytic reagent composition may include one or more detergents which may be present in an amount from about 1% to about 15%, for example from about 2% to about 12%, for example from about 4% to about 10%, for example about 5%. The detergent may be any detergent suitable for the purpose intended herein. In some embodiments, the detergent may include sodium dodecyl sulfate (SDS), lithium dodecyl sulfate (LDS), sodium taurodeoxycholate (NaTDC), sodium taurocholate (NaTC), sodium glycocholate (NaGC), sodium deoxycholate (NaDC), sodium cholate, sodium alkylbenzene sulfonate (NaABS), Na N-lauroyl sarcosine (NLS), salts of carboxylic acids (i.e., soaps), salts of sulfonic acids, salts of sulfuric acid, phosphoric and polyphosphoric acid esters, alkylphosphates, monoalkyl phosphate (MAP), and salts of perfluorocarboxylic acids, anionic detergents

The pre-analytic reagent composition may include acetic acid which may be present in an amount from about 6% to about 11%, preferably from about 5% to about 10%, for example from about 6% to about 8%, for example about 6%.

In some embodiments according to the disclosure, the sample is contacted with the composition at room temperature for about 15 minutes to about 2 hours or for about 15 minutes to about 1 hour or for about 15 to about 30 minutes. In some embodiments, the sample is contacted with the composition at room temperature for about 2 hours or for about 1 hour or for about 30 minutes.

In some embodiments according to the disclosure, the sample is mixed with the pre-analytic reagent in a 2:1 to 1:2 ratio (v/v) or in a 1,5:1 to 1:1,5 ratio (v/v). In some embodiments, the sample is mixed with the pre-analytic reagent in a 1:1 ratio (v/v).

The term “about” as used herein in the context of a stated concentrations of solutions may include exactly the stated concentration, and also include a concentration difference of 1, 2, or a fraction of the unit of measurement, for example, in mM of 1 mM, 2 mM, or a fraction thereof, plus or minus the stated concentration, or, for example, in % of 1%, 2%, or a fraction thereof, plus or minus the stated concentration.

The term “completely inactivate” refers to the process of completely destroying or killing Mycobacteria in a sample using chemical means thereby rendering Mycobacteria inactive and/or unable to replicate.

TABLE 1

| Pre-analytic Reagent #1 | Concentration |
|---|---|
| guanidine thiocyanate | 4M |
| sodium citrate | 50 mM |
| Dithiothreitol | 130 mM |
| polydocanol | 5% |
| acetic acid | 6% |
| Na N-lauroyl sarcosine | 1% |

Embodiments of the present disclosure will be further described in the following examples.

### EXAMPLES

The following examples are provided to aid the understanding of the present subject matter, the true scope of which is set forth in the appended claims. It is understood that modifications can be made in the procedures set forth.

### Example I

### Raw Sputum Liquefaction

### Materials and Method

Raw sputum sample (MTB culture negative). Sample was transparent and had very gelatinous consistency.

Pre-analytic reagent #1 (Table 1)

MTB negative raw sputum sample was mixed in 1:1 ratio with the pre-analytic reagent then vortexed for approximately 10 seconds and left to stand at room temperature for approximately 20 minutes. The extent of liquefaction of sputum was assessed by visual inspection and the ability to draw the liquefied specimen through a thin-tipped transfer pipette.

### RESULTS

After the approximately 20 minute room temperature incubation with the pre-analytic reagent, the liquefaction of sputum was successfully achieved.
Some bubbles were present after initial vortex, but bubbles dissipated during room temperature incubation time. After incubation time, sputum mixture appeared liquefied (i.e., no longer gelatinous, no visual chunks) based on visual inspection. The liquefied sample was easily pipetted through a thin-tip transfer pipette.

### Example II

### Mycobacterium bovis BCG Inactivation

### Materials and Method

M. bovis BCG cell stock, Lowenstein Jensen agar slant (Hardy Flask): LJ slants

PBS (Phosphate buffer saline), Pre-analytic reagent #1 (Table 1)

For inactivation assessment, Mycobacterium bovis BCG cell stock at a high concentration (estimated to be >1e8 CFU/ml) was mixed with the pre-analytic reagent, vortexed for approximately 10 seconds, and then incubated at room temperature for approximately 20 minutes. For the positive control, cells were only treated with PBS and followed by vortexing and room temperature incubation. After incubation, the treated specimens as well as un-treated positive controls were centrifuged at >10,000 rpm for 1-2 minutes to pellet any potential surviving cells. After centrifugation, the supernatant was removed and the pellet was washed once by resuspending in PBS buffer and subsequent centrifugation. After the wash, the pellet was resuspended in 100 uL of PBS and plated onto Lowenstein-Jensen slants (LJ-slants) to assess the efficacy of the inactivation procedure. Slants were incubated at 37° C., 5% CO₂, for 8 weeks to evaluate for colony formation.

### RESULTS

Cells treated with the pre-analytic reagent did not exhibit any growth after 8 weeks of incubation. Growth was observed after approximately 2-3 weeks for the positive controls on the LJ slants plated with PBS treated cells.

While the foregoing subject matter has been described in some detail for purposes of clarity and understanding, it will be clear to one skilled in the art from a reading of this disclosure that various changes in form and detail can be made. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A method for liquefaction of a sputum sample and inactivation of Mycobacteria suspected of being contained therein, comprising
contacting a sputum sample suspected of containing Mycobacteria with an amount of a composition effective to liquefy the sputum sample and to completely inactivate the Mycobacteria at room temperature,
said composition comprising:
a chaotropic agent;
a chelator;
a reducing agent;
a detergent; and
acetic acid.

2. The method of claim 1, wherein:
the chaotropic agent is present in an amount from about 0.5 M to about 6 M;
the chelator is present in an amount from about 0.01 mM to about 400 mM;
the reducing agent is present in an amount from about 130 mM to about 650 mM;
the detergent is present in an amount from about 0.2% to about 15%; and
the acetic acid is present in an amount from about 6% to about 11%.

3. The method of claim 1, wherein the chaotrope is guanidine thiocyanate (GuSCN).

4. The method of claim 1, wherein the chelator is sodium citrate.

5. The method of claim 1, wherein the reducing agent is Dithiothreitol (DTT).

6. The method of claim 1, wherein the detergent is polydocanol and Na N-lauroyl sarcosine.

7. The method of claim 1, wherein the Mycobacteria is Mycobacterium tuberculosis (MTB).

8. A pre-analytic reagent for liquefaction of a sputum sample and inactivation of one or more Mycobacteria suspected of being contained therein, comprising:
a chaotropic agent;
a chelator;
a reducing agent;
a detergent; and
acetic acid,
wherein the pre-analytic reagent is effective to liquefy the sputum sample and completely inactivate the Mycobacteria at room temperature.

9. The pre-analytic reagent of claim 8, wherein:
the chaotropic agent is present in an amount from about 0.5 M to about 6 M;
the chelator is present in an amount from about 0.01 mM to about 400 mM;
the reducing agent is present in an amount from about 130 mM to about 650 mM;
the detergent is present in an amount from about 0.2% to about 15%; and
the acetic acid is present in an amount from about 6% to about 11%.

10. The pre-analytic reagent of claim 8, wherein the chaotrope is guanidine thiocyanate (GuSCN).

11. The pre-analytic reagent of claim 8, wherein the chelator is sodium citrate.

12. The pre-analytic reagent of claim 8, wherein the reducing agent is Dithiothreitol (DTT).

13. The pre-analytic reagent of claim 8, wherein the detergent is polydocanol and Na N-lauroyl sarcosine.

14. The pre-analytic reagent of claim 8, wherein the Mycobacteria is Mycobacterium tuberculosis (MTB).

15. A kit, comprising a composition effective to liquefy a sputum sample and completely inactivate Mycobacteria suspected to be contained therein at room temperature, the composition comprising:
a chaotropic agent;
a chelator;
a reducing agent;
a detergent; and
acetic acid.

16. The kit of claim 15, wherein:
the chaotropic agent is present in an amount from about 0.5 M to about 6 M;
the chelator is present in an amount from about 0.01 mM to about 400 mM;
the reducing agent is present in an amount from about 130 mM to about 650 mM;
the detergent is present in an amount from about 0.2% to about 15%; and
the acetic acid is present in an amount from about 6% to about 11%.

17. The kit of claim 15, wherein the chaotrope is guanidine thiocyanate (GuSCN).

18. The kit of claim 15, wherein the chelator is sodium citrate.

19. The kit of claim 15, wherein the reducing agent is Dithiothreitol (DTT).

20. The kit of claim 15, wherein the detergent is polydocanol and Na N-lauroyl sarcosine.
